(19) United States
(12) Patent Application Publication
(10) Pub. No.: US 2015/0313793 A1
(43) Pub. Date: Nov. 5, 2015

(54) MIRROR THERAPY DEVICE

(71) Applicants: Hsin-Min Lee, Tainan City (TW); Ping-Chia Li, Kaohsiung City (TW)

(72) Inventors: Hsin-Min Lee, Tainan City (TW); Ping-Chia Li, Kaohsiung City (TW)

(21) Appl. No.: 14/265,841

(22) Filed: Apr. 30, 2014

(51) Int. Cl. A61H 99/00; A61F 2/58

(52) U.S. Cl. CPC A61H 99/00; A61F 2/583; A61F 2002/5058

(57) ABSTRACT

A mirror therapy device includes a support unit, a camera unit and a display unit. The support unit has a control area adapted for supporting a normal hand of a patient thereon, and an image area disposed adjacent to the control area. The camera unit is disposed above the support unit, and includes a lens confronting the control area and adapted for capturing an image of the normal hand, and a processor coupled electrically to the lens for receiving the image thus captured and generating a mirror image mirroring the captured image. The display unit is disposed on the image area and is connected electrically to the processor for receiving and displaying the mirror image.

### BACKGROUND OF THE INVENTION

1. Field of the Invention

The invention relates to a mirror therapy device, more particularly to a mirror therapy device for rehabilitating an impaired hand of a human patient.

2. Description of the Related Art

Referring to FIG. 1, a conventional mirror therapy device 1 is mainly designed to include a one-way mirror 12 placed on a surface 111 of a table 11. Before rehabilitation training, a patient must sit at the table 11, placing both the normal and impaired hands 21, 22 on the surface 111 of the table 11 at respective opposite sides of the one-way mirror 12 such that the normal hand 21 confronts the mirror side 121 of the one-way mirror 12. During rehabilitation, when the normal hand 21 moves in front of the one-way mirror 12, a mirror image 23 thereof is shown in the mirror side 121 and extends in the same direction as the impaired hand 22; therefore, when the patient sees the movement of the normal hand 21 through the mirror image 23, the brain will trigger the impaired hand 22 to attempt to imitate the same movement, thus achieving rehabilitation training of the impaired hand 22.

However, due to the patient's hands 21, 22 being placed on two opposite sides of the one-way mirror 12 and since the patient must not see his/her impaired hand 22, the patient has to tilt his/her head to a position where the impaired hand 22 is fully out of sight while focusing on the movements of the normal hand 21 appearing in the mirror image 23 on the mirror side 121 of the one-way mirror 12. The position of the one-way mirror 12 must also be adjusted relative to the patient's eyesight. As such, after a long period of rehabilitation training, the patient is prone to feel pain and/or discomfort at his/her shoulder and neck areas.

### SUMMARY OF THE INVENTION

Therefore, the object of the present invention is to provide a mirror therapy device that can eliminate the aforesaid drawbacks of the prior art.

According to the present invention, there is provided a mirror therapy device adapted for rehabilitation of an impaired hand of a human patient. The mirror therapy device includes a support unit, a camera unit and a display unit. The support unit has a control area adapted for supporting a normal hand of the patient thereon, and an image area disposed adjacent to the control area. The camera unit is disposed above the support unit, and includes a lens and a processor. The lens confronts the control area and is adapted for capturing an image of the normal hand of the patient. The processor is coupled electrically to the lens for receiving therefrom the image captured thereby and generating a mirror image mirroring the image captured by the lens. The display unit is disposed on the image area of the support unit and connected electrically to the processor for receiving and displaying the mirror image.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of a conventional mirror therapy device in use;

FIG. 2 is a perspective view of a preferred embodiment of a mirror therapy device according to the present invention, where the dashed lines illustrate a camera unit adjustable via operation of an adjustment unit; and

FIGS. 3 and 4 illustrate the preferred embodiment in use.

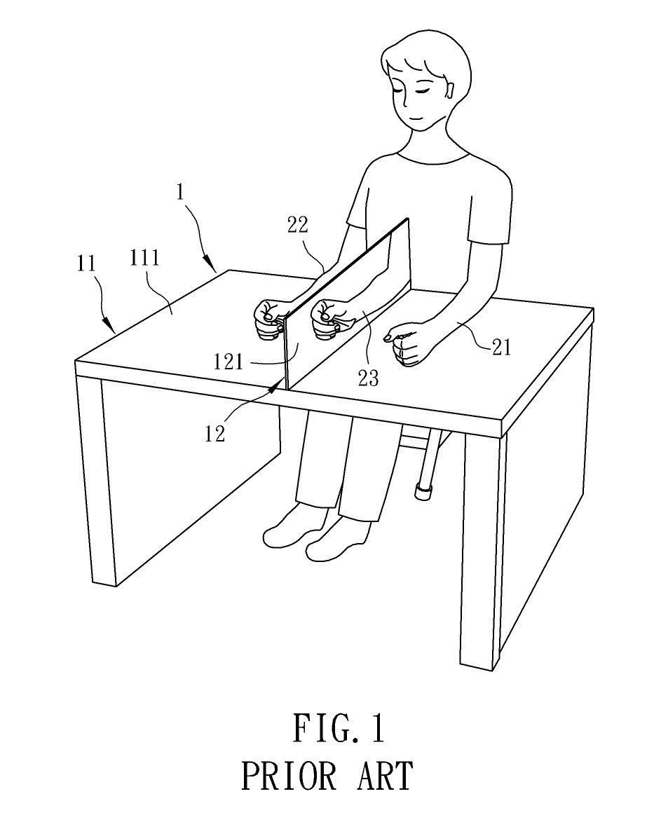

### DETAILED DESCRIPTION OF THE PREFERRED EMBODIMENT

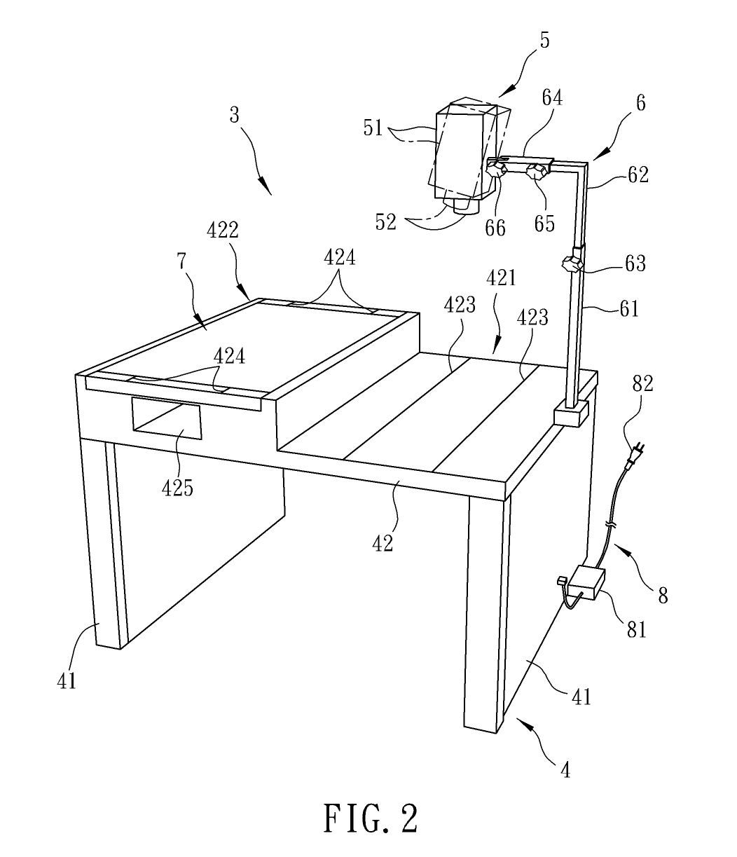

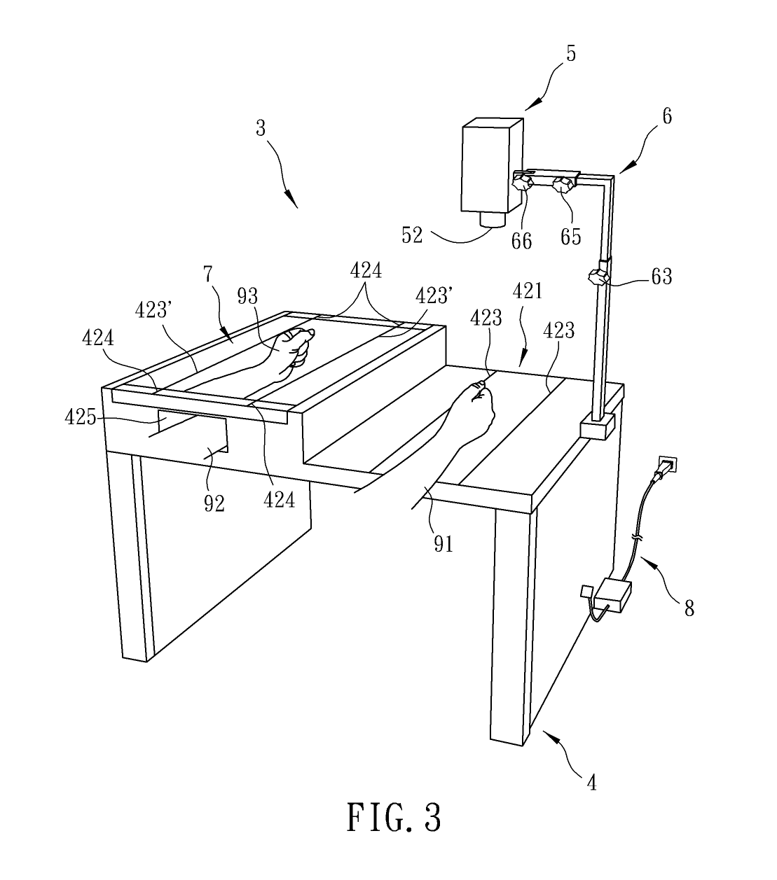

Referring to FIGS. 2 and 3, the preferred embodiment of a mirror therapy device 3 according to the present invention is adapted for rehabilitating an impaired hand 92 of a human patient. The mirror therapy device 3 includes a support unit 4, a camera unit 5, an adjustment unit 6, a display unit 7 and a power supply unit 8.

The support unit 4 includes two support legs 41, and a support plate 42 mounted on top of the support legs 41. The support plate 42 has a control area 421 adapted for supporting a normal hand 91 of the patient thereon, and an image area 422 disposed adjacent and protruding relative to the control area 421. The control area 421 is formed with a plurality of parallel and equally-spaced partitioning lines 423 for dividing the control area 421 into a plurality of subareas. The display unit 7 is disposed on the image area 422. The image area 422 has two end sections disposed at opposite sides of the display unit 7. Each of the end sections is formed with a plurality of parallel and equally-spaced partitioning line segments 424 extending parallel to the partitioning lines 423 for dividing the end section into a plurality of subsections that correspond in number to the subareas of the control area 421. The partitioning line segments 424 of one of the end sections are aligned respectively with those of the other of the end sections. In this embodiment, the control area 421 is formed with two of the partitioning lines 423 dividing the control area 421 into three subareas, and each end section of the image area 422 is formed with two of the partitioning line segments 424 dividing each end section into three subsections. In addition, the support unit 4 further has a rehabilitation area 425 formed in the support plate 42, disposed under the image area 422 and adapted for receiving the impaired hand 92 of the patient for keeping the impaired hand 92 out of the patient's sight.

The camera unit 5 is disposed above the support unit 4, and includes a main body 51, a lens 52 and a processor (not shown). The lens 52 confronts the control area 421 and is adapted for capturing an image of the normal hand 91 of the patient. The processor is disposed in the main body 51 and is coupled electrically to the lens 52 for receiving therefrom the image captured thereby and generating a mirror image 93 mirroring the image captured by the lens 52.

The adjustment unit 6 is connected to and between the support unit 4 and the camera unit 5 for adjusting position and orientation of the camera unit 5 relative to the support unit 4. The adjustment unit 6 includes a fixing bracket 61, an extending bracket 62, a first locking member 63, a horizontal bracket 64, a second locking member 65 and a third locking member 66. The fixing bracket 61 is fastened detachably on the support plate 42. The extending bracket 62 is connected to the fixing bracket 61 and is vertically and telescopically movable relative to the fixing bracket 61. The first locking member 63 is used for locking the extending bracket 62 to the fixing bracket 61. The horizontal bracket 64 is connected to the extending bracket 62 and is horizontally and telescopically movable relative to the extending bracket 62. The second locking member 65 is used for locking the horizontal bracket 64 to the extending bracket 62. The camera unit 5 is connected pivotally to a distal end of the horizontal bracket 64 that is distal from the extending bracket 62, and the third locking member 66 is operable for enabling and disabling the pivoting of the camera unit 5 relative to the horizontal bracket 64.

The display unit 7 is connected electrically to the processor of the camera unit 5 for receiving and displaying the mirror image 93. In this embodiment, the display unit 7 is a tablet-type display device.

The power supply unit 8 is coupled electrically to the camera unit 5 and the display unit 7, and includes a transformer 81 and a plug 82. The transformer 81 is coupled electrically to the camera unit 5 and the display unit 7, and the plug 82 is adapted to interconnect electrically the transformer 81 and an external power source.

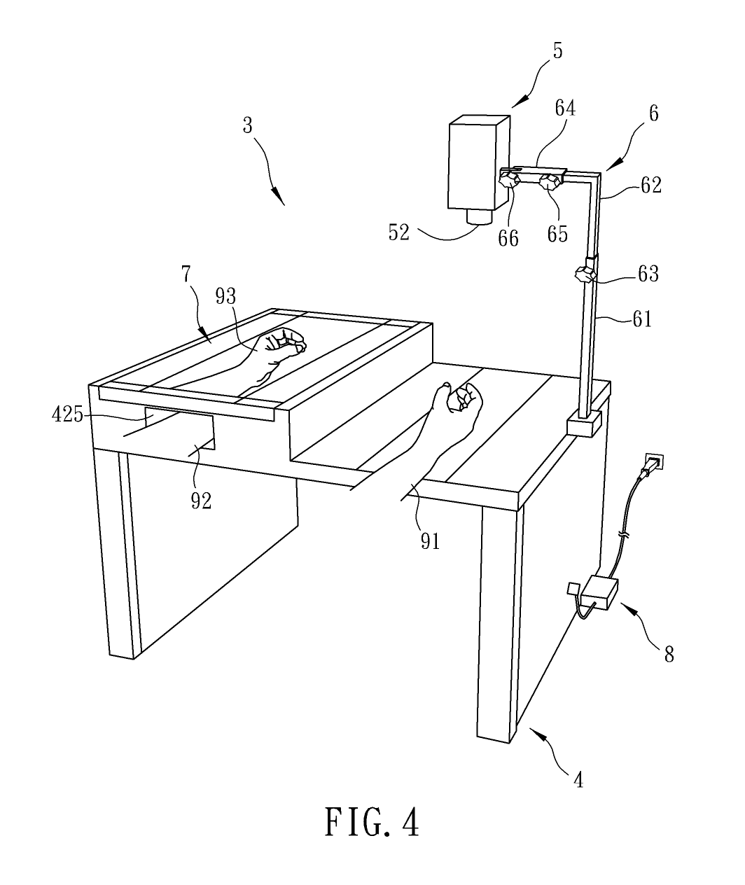

Referring to FIGS. 3 and 4, the mirror therapy device 3 is used for rehabilitation training by the patient whose one hand 92 has been weakened or disabled as a result of stroke, disease or accident. When in use, the partitioning lines 423 serve as guidance in the correct positioning of the normal hand 91, where the normal hand 91 of the patient is placed on the middle subarea of the control area 421 of the support plate 42 (that is, the central one of the three equal subareas of the control area 421 formed by the partitioning lines 423), and the impaired hand 92 is placed into the rehabilitation area 425. A physical therapist then adjusts the height of the camera unit 5 via the first locking member 63, the horizontal positioning of the camera unit 5 via the second locking member 65 and the angle of the camera unit 5 via the third locking member 66 such that the lens 52 of the camera unit 5 is appropriately positioned to capture an image of the normal hand 91, as well as the partitioning lines 423, during movement of the normal hand 91. The processor of the camera unit 5 receives the image captured by the lens 52 and generates the mirror image 93, which is then outputted by the processor thereon to the display device 7 for display. When the mirror image 93 is shown in the display device 7, each mirrored partitioning line 423′ is registered with a respective pair of the partitioning line segments 424 of the opposite end sections of the image area 422 such that the overall view of the image area 422 resembles a mirrored view of the entire control area 421. At this moment, since the impaired hand 92 is out of the patient's sight, the patient sees the mirror image 93 instead of the impaired hand 92. This visual feedback transmits a message to the patient's brain, triggering the impaired hand 92 (currently out of sight) to attempt to imitate the movement of the normal hand 91 shown in the mirror image 93, helping the impaired hand 92 to move and eventually recover. Due to the design of this invention, the patient can easily and comfortably view the mirror image 93 without awkwardly tilting his/her head and using uncomfortable positions for rehabilitation training. Therefore, the purpose of this invention is served.

It is worth mentioning herein that, with the corresponding setup between the partitioning lines 423 and the partitioning line segments 424, and the image-capturing of the normal hand 91 along with the partitioning lines 423, as well as the display of the mirror image 93 containing mirrored partitioning lines 423′, appropriate positioning of the normal hand 91 is facilitated for the mirror image 93 to be displayed in the display device 7 in such a manner that the mirrored normal hand smoothly extends from an exposed arm portion of the patient's impaired hand 82. Thus the patient will see two normal hands to lead the patient's brain to believe that the mirrored normal hand 91 in the mirror image 93 shown on the display device 7 is his/her impaired hand 92, which will consequently train the brain to control synchronous movement of the impaired hand 92 with the normal hand 91.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A mirror therapy device adapted for rehabilitation of an impaired hand of a human patient, said mirror therapy device comprising:
a support unit having a control area that is adapted for supporting a normal hand of the patient thereon, and an image area that is disposed adjacent to said control area;
a camera unit disposed above said support unit, and including
a lens that confronts said control area and that is adapted for capturing an image of the normal hand of the patient, and
a processor that is coupled electrically to said lens for receiving therefrom the image captured thereby and generating a mirror image mirroring the image captured by said lens; and
a display unit disposed on said image area of said support unit and connected electrically to said processor for receiving and displaying the mirror image.

2. The mirror therapy device as claimed in claim 1, wherein said support unit includes at least two support legs and a support plate mounted on top of said support legs and having said control area and said image area.

3. The mirror therapy device as claimed in claim 2, wherein said support unit further has a rehabilitation area formed in said support plate, disposed under said image area and adapted for receiving the impaired hand of the patient and keeping the impaired hand out of the patient's sight.

4. The mirror therapy device as claimed in claim 3, wherein:
said control area is formed with a plurality of parallel and equally-spaced partitioning lines for dividing said control area into a plurality of subareas; and
said image area has two end sections disposed at opposite sides of said display unit, each of said end sections being formed with a plurality of parallel and equally-spaced partitioning line segments that extend parallel to said partitioning lines for dividing said end section into a plurality of subsections which correspond in number to said subareas of said control area, said partitioning line segments of one of said end sections being aligned respectively with those of the other one of said end sections.

5. The mirror therapy device as claimed in claim 4, wherein:
said control area is formed with two of said partitioning lines that divide said control area into three of said subareas; and
each of said end sections of said image area is formed with two of said partitioning line segments that divide said end section into three of said subsections.

6. The mirror therapy device as claimed in claim 4, further comprising an adjustment unit connected to and between said support unit and said camera unit for adjusting position and orientation of said camera unit relative to said support unit.

7. The mirror therapy device as claimed in claim 6, wherein said adjustment unit includes:
a fixing bracket fastened detachably on said support plate;
an extending bracket connected to said fixing bracket and vertically and telescopically movable relative to said fixing bracket;
a first locking member for locking said extending bracket to said fixing bracket;
a horizontal bracket connected to said extending bracket and horizontally and telescopically movable relative to said extending bracket, said camera unit being connected pivotally to a distal end of said horizontal bracket; and
a second locking member for locking said horizontal bracket to said extending bracket.

8. The mirror therapy device as claimed in claim 7, wherein said adjustment unit further includes a third locking member operable for enabling and disabling pivot action of said camera unit relative to said horizontal bracket.

9. The mirror therapy device as claimed in claim 5, further comprising an adjustment unit connected to and between said support unit and said camera unit for adjusting position and orientation of said camera unit relative to said support unit.

10. The mirror therapy device as claimed in claim 9, wherein said adjustment unit includes:
a fixing bracket fastened detachably on said support plate;
an extending bracket connected to said fixing bracket and vertically and telescopically movable relative to said fixing bracket;
a first locking member for locking said extending bracket to said fixing bracket;
a horizontal bracket connected to said extending bracket and horizontally and telescopically movable relative to said extending bracket, said camera unit being connected pivotally to a distal end of said horizontal bracket; and
a second locking member for locking said horizontal bracket to said extending bracket.

11. The mirror therapy device as claimed in claim 10, wherein said adjustment unit further includes a third locking member operable for enabling and disabling pivot action of said camera unit relative to said horizontal bracket.

12. The mirror therapy device as claimed in claim 1, further comprising a power supply unit coupled electrically to said camera unit and said display unit.

13. The mirror therapy device as claimed in claim 12, wherein said power supply unit includes a transformer that is coupled electrically to said camera unit and said display unit, and a plug that is adapted to interconnect electrically said transformer and an external power source.
